(19) United States
(12) Patent Application Publication
(10) Pub. No.: US 2007/0250143 A1
(43) Pub. Date: Oct. 25, 2007

(54) MULTI-CONDUCTOR RIBBON COILED MEDICAL DEVICE LEAD

(76) Inventor: John L. Sommer, Coon Rapids, MN (US)

Correspondence Address: MEDTRONIC, INC., 710 MEDTRONIC PARKWAY NE, MINNEAPOLIS, MN, 55432-9924, US

(21) Appl. No.: 11/379,334

(22) Filed: Apr. 19, 2006

(51) Int. Cl. A61N 1/05

(52) U.S. Cl. 607/116

(57) ABSTRACT

A medical device lead that includes a first insulative material and a plurality of substantially parallel conductive filars extending from a proximal end to a distal end within the first insulative material. The first insulative material and the plurality of conductive filars are coiled to a define a lumen, and a first opening is formed by the first insulative material spaced distally from a second opening formed by the first insulative material, the first opening exposing a first conductive filar of the plurality of conductive filars within the lumen and the second opening exposing a second conductive filar of the plurality of conductive filars at a predetermined location relative to the first opening within the lumen. In this way, the first opening is positioned adjacent to a proximally extending tab associated with a first electrode of the plurality of electrodes and the second opening is simultaneously positioned adjacent to a proximally extending tab associated with a second electrode of the plurality of electrodes when the electrode assembly is positioned within the lumen.

### TECHNICAL FIELD

The invention relates to medical devices and, more particularly, to implantable medical device leads for use with implantable medical devices (IMDs).

### BACKGROUND

In the medical field, implantable leads are used with a wide variety of medical devices. For example, implantable leads are commonly used to form part of implantable cardiac pacemakers that provide therapeutic stimulation to the heart by delivering pacing, cardioversion or defibrillation pulses. The pulses can be delivered to the heart via electrodes disposed on the leads, e.g., typically near distal ends of the leads. In that case, the leads may position the electrodes with respect to various cardiac locations so that the pacemaker can deliver pulses to the appropriate locations. Leads are also used for sensing purposes, or for both sensing and stimulation purposes.

In addition, implantable leads are used in neurological devices such as deep-brain stimulation devices and spinal cord stimulation devices. For example, leads may be stereotactically probed into the brain to position electrodes for deep brain stimulation. Leads are also used with a wide variety of other medical devices including, for example, devices that provide muscular stimulation therapy, devices that sense chemical conditions in a patient's blood, gastric system stimulators, implantable nerve stimulators, implantable lower colon stimulators, e.g., in graciloplasty applications, implantable drug or beneficial agent dispensers or pumps, implantable cardiac signal loops or other types of recorders or monitors, implantable gene therapy delivery devices, implantable incontinence prevention or monitoring devices, implantable insulin pumps or monitoring devices, and the like. In short, medical leads may be used for sensing purposes, stimulation purposes, sensing incident to drug delivery, and the like.

### BRIEF DESCRIPTION OF DRAWINGS

Aspects and features of the present invention will be appreciated as the same becomes better understood by reference to the following detailed description of the embodiments of the invention when considered in connection with the accompanying drawings, wherein:

FIG. 1 is a perspective view of a portion of a ribbon-like structure that can be formed according to an embodiment of the invention as part of a medical lead manufacturing process;

FIG. 2 is another perspective view illustrating the ribbon-like structure of FIG. 1 coiled to form a lumen;

FIG. 3 is a cross-sectional front view of a ribbon-like structure including two insulated filars disposed substantially parallel to one another within an insulative material;

FIG. 4 is a cross-sectional front view of a ribbon-like structure including two non-insulated filars disposed substantially parallel to one another within an insulative material;

FIG. 5 is a cross-sectional front view of a ribbon-like structure including three filars disposed substantially parallel to one another within an insulative material;

FIG. 6 is a cross-sectional front view of a ribbon-like structure including four filars disposed substantially parallel to one another within an insulative material;

FIG. 7 is a cross-sectional front view of a ribbon-like structure including two filars and a fiber core disposed within an insulative material;

FIG. 8 is a cross-sectional side view of a portion of a medical lead including a ribbon-like structure coiled to define a lumen;

FIG. 9 is another cross-sectional side view of a portion of a medical lead including a ribbon-like structure coiled to define a lumen;

FIG. 10 is another cross-sectional side view of a portion of a medical lead including ring electrodes that provide electrical contact surfaces to the lead;

FIG. 11 is a schematic diagram of a coil conductor of a medical device positioned on an electrode assembly according to an embodiment of the present invention;

FIG. 12 is a partial schematic diagram of a coil conductor positioned on an electrode assembly according to an embodiment of the present invention; and

FIG. 13 is a conceptual perspective view of a medical device system including a medical device coupled to a lead according to an embodiment of the present invention.

### DETAILED DESCRIPTION

The present invention is directed to a multi-filar coiled medical lead, techniques for manufacturing such a lead, and systems that include a medical device coupled to a multi-filar coiled medical lead according to the present invention. In particular, the multi-filar coiled medical lead of the present invention includes multiple filars embedded in an insulative material. The filars and insulative material are coiled to define a lumen of the lead. In other words, the filars are embedded in the insulative material to form a ribbon-like structure, and the ribbon-like structure is coiled to define a lumen of the medical lead. The coiled ribbon-like structure is insertable within a lead body, and electrodes are electrically coupled to the filars extending through the lead body and insulative material. The lead may find useful application in any of a wide variety of implantable medical lead applications, including stimulation or sensing leads used with cardiac pacemakers or similar devices, high voltage stimulation leads, neurological stimulation leads such as deep brain stimulation (DBS) leads, and the like.

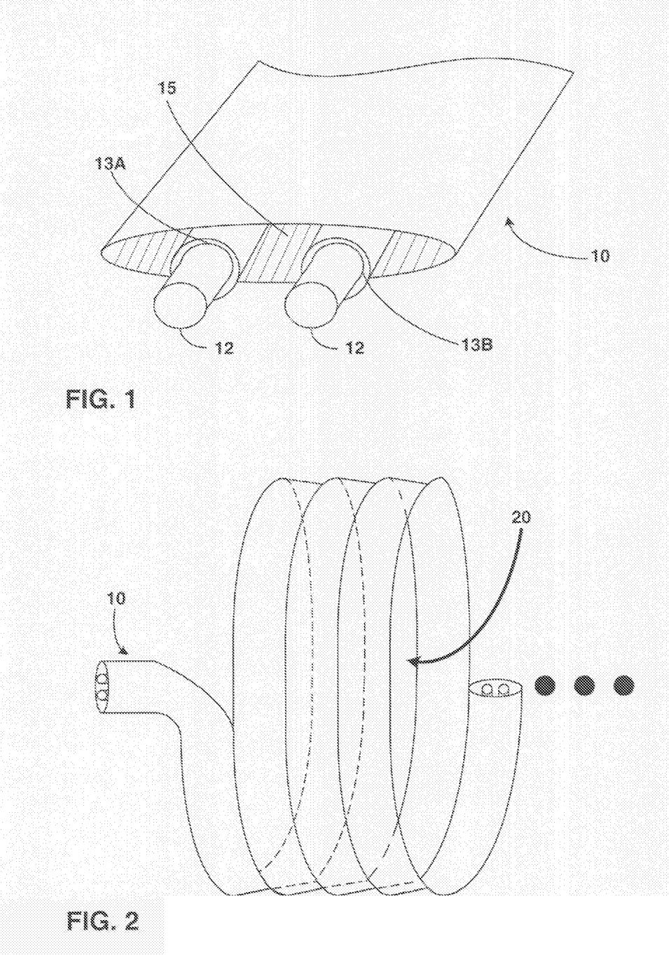

FIG. 1 is a perspective view of a portion of a ribbon-like structure 10 that can be formed according to an embodiment of the invention as part of a medical lead manufacturing process. As illustrated in FIG. 1, a medical lead according to an embodiment of the present invention includes a ribbon-like structure 10 formed with two or more conductive filars 12 embedded in an insulative material 15. Any number of filars may be used in accordance with the invention. In general, the filars 12 are disposed within insulative material 15 substantially parallel to one another. Conductive filars 12 are formed from any desired conductive material, selected based on the application of the lead being manufactured. For example, conductive filars 12 may be formed from silver, platinum, gold, copper, a conductive alloy, or any other conductive material suitable for use in a medical lead. In some cases, different conductive materials may be used for different ones of filars 12.

According to an embodiment of the present invention, conductive filars 12 are formed as insulated conductive filars by including an additional insulative cladding 13A, 13B that is formed on conductive filars 12 prior to embedding filars 12 in insulative material 15. Insulative cladding 13A, 13B may be formed to have different colors in order to enable one of filars 12 to be distinguishable from another of filars 12. Alternatively or additionally, insulative material 15 may also be color-coded to distinguish filars 12 from one another. In both cases, insulative cladding 13A, 13B provides redundant insulation to the conductive cores of filers 12, in addition to the insulation provided by insulative material 15.

An extrusion process may be used to create ribbon-like structure 10. In particular, conductive filars 12 are disposed substantially parallel to one another and insulative material 15 is extruded about the parallel filars 12.

In some production processes, a polymer wound conductor coil is subjected to annealing temperatures of the coated polymer material to either strain relieve the molded polymer or shape the polymer coil in any given shape. For example, thermal shaping of the wound coil may be achieved by heating the shaped coil (on a shaped mandrel) to an annealing temperature of material 15. Shaping of the wound coil can aid in maintaining the lead shape for lead assembly and subsequent implant. For example, a J-shaped, curved distal end of the pacing lead may be desirable for placement in a patient's atrium.

Extrusion of insulative material 15 about filars 12 provides an opportunity to wind the coil while insulative material 15 is in a heated state subsequent to exiting the extruder. Accordingly, following extrusion, filers 12 are wound into coils as insulative material 15 cools down. Such a technique improves assembly of a multi-filer coiled medical lead.

Insulative material 15 may be formed from any of a wide variety of insulative materials such as polyimide, urethane, silicone, tetrafluroethylene (ETFE), polytetrafluroethylene (PTFE), or the like. If filars 12 include insulative cladding 13A, 13B, the insulative cladding 13A, 13B may similarly be formed from the same polyimide, urethane, silicone, tetrafluroethylene (ETFE), polytetrafluroethylene (PTFE) material, or the like.

FIG. 2 is a perspective view of a multi-conductor ribbon coiled lead according to an embodiment of the present invention. As illustrated in FIG. 2, ribbon-like structure 10 is coiled to define a lumen 20. By grouping multiple filars together in the insulative material, manufacture of medical leads is simplified because the multiple filars can be collectively coiled. The coiled ribbon-like structure 10 illustrated in FIG. 2 is then inserted into a lead body to form a medical lead, and one or more electrodes are subsequently electrically coupled to the filars as outlined in greater detail below.

In order to form the coil in ribbon-like structure 10, an inner core may be used and then removed. For example, ribbon-like structure 10 may be coiled about a cylindrical core or mandrel. After such coiling is completed, the cylindrical core is then removed such that coiled ribbon-like structure 10 defines lumen 20. Thus, the size of the cylindrical core defines the size of lumen 20. Coiled ribbon-like structure 10 is inserted into a lead body before or after removing the cylindrical core that defines the size of lumen 20. Other coiling techniques may also be used. The coiling may be performed in either direction, e.g., clockwise or counter clockwise. In other words, the coil wrap direction can be either right or left, i.e., in the Z or S direction

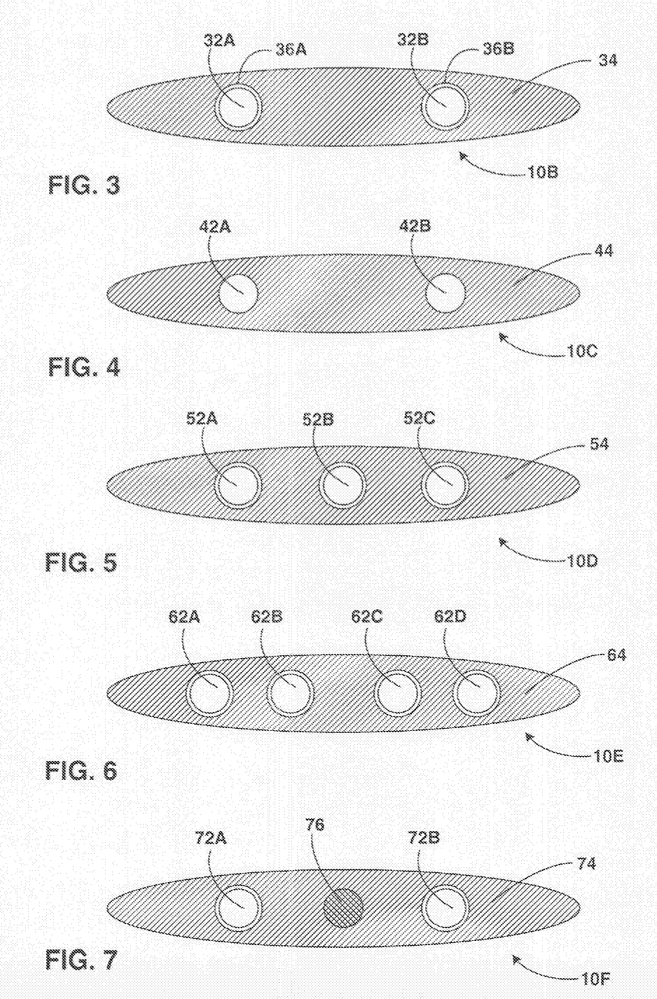

FIG. 3 is a cross-sectional front view of ribbon-like structure 10B according to an embodiment of the present invention including two filars 32A, 32B disposed substantially parallel to one another within insulative material 34. As illustrated in FIG. 3, filars 32A and 32B may include additional insulative cladding 36A, 36B, respectively, formed about a conductive core.

FIG. 4 is another cross-sectional front view of ribbon-like structure 10C including two filars 42A, 42B disposed substantially parallel to one another within insulative material 44. Filars 42A and 42B do not include additional insulative cladding. In other words, in FIG. 4, filars 42A, 42B are formed as conductive wires that do not include insulative cladding, but are embedded in insulative material 44, e.g., via an extrusion process.

According to the present invention, any number of filars may be embedded in an insulative material to define a ribbon-like structure in accordance with the invention. In some cases, ten or more filars may be disposed substantially parallel to one another to define the ribbon-like structure, which can be coiled to define a lumen and inserted into a lead body.

FIG. 5 is a cross-sectional front view of ribbon-like structure 10D including three filars 52A, 52B, 52C disposed substantially parallel to one another within insulative material 54. FIG. 6 is a cross-sectional front view of ribbon-like structure 10E including four filars 62A, 62B, 62C, 62D disposed substantially parallel to one another within insulative material 64.

FIG. 7 is a cross-sectional front view of ribbon-like structure 10F including two filars 72A, 72B disposed substantially parallel to one another within insulative material 74. In addition, a rigid material such as fiber core 76 is also disposed within insulative material 78 substantially parallel to filars 72. Fiber core 76 or other rigid material may improve the strength, stiffness or other mechanical characteristics of ribbon-like structure 10F. Accordingly, by selecting a particular fiber core 76 (or number of cores), desirable mechanical characteristics for a medical lead can be defined. As one example, polyester fiber could be used to realize fiber core 76.

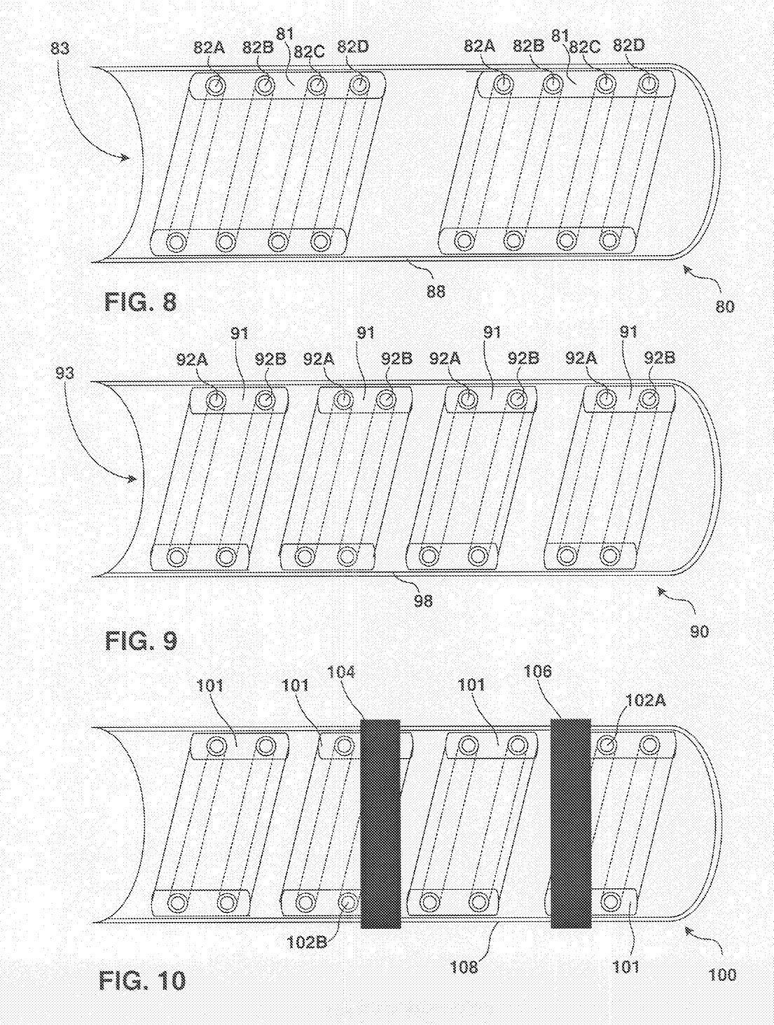

FIG. 8 is a cross-sectional side view of a portion of a medical lead 80 including a ribbon-like structure 81 coiled to define a lumen 83. Ribbon-like structure 81 includes four filars 82A, 82B, 82C and 82D disposed substantially parallel to one another within insulative material 84. Ribbon-like structure 81 is coiled to define lumen 83 and inserted within lead body 88. Lead body 88 may include, for example, an insulative tubing formed of polyimide, urethane, silicone, tetrafluroethylene (ETFE), polytetrafluroethylene (PTFE), or the like.

FIG. 9 is another cross-sectional side view of a portion of a medical lead 90 including a ribbon-like structure 91 coiled to define a lumen 93. Ribbon-like structure 91 includes two filars 92A and 92B disposed substantially parallel to one another within insulative material 94. Ribbon-like structure 91 is coiled to define lumen 93 and inserted within lead body 98. Lead body 98 may include an insulative tubing formed of polyimide, urethane, silicone, tetrafluroethylene (ETFE), polytetrafluroethylene (PTFE) material, or the like.

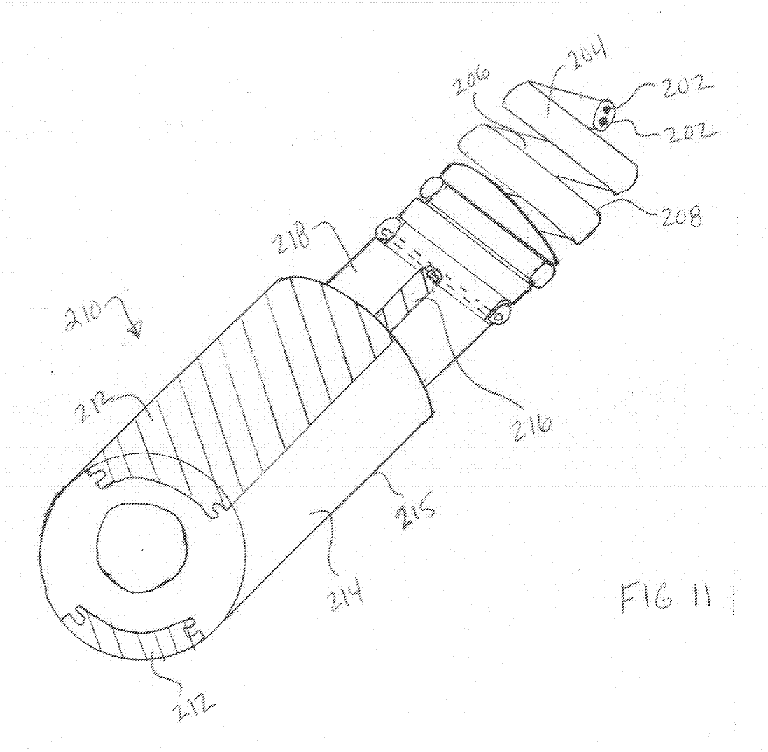

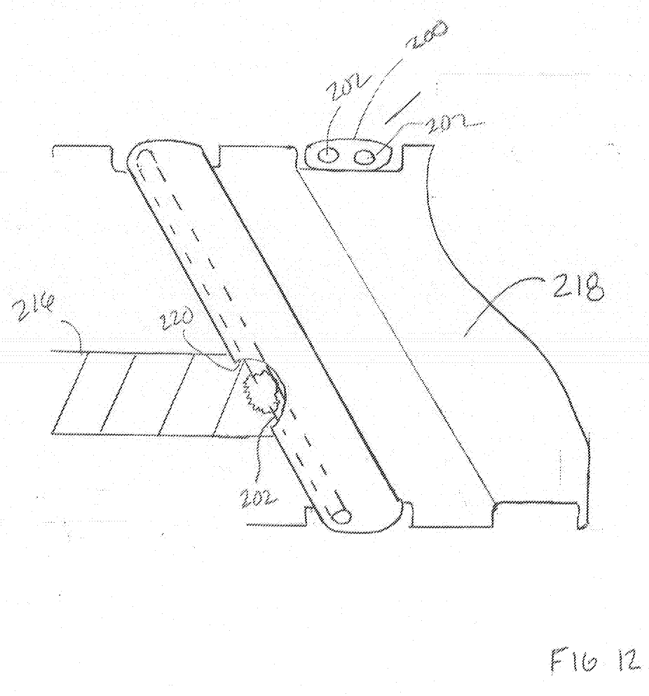

FIG. 10 is another cross-sectional side view of a portion of a medical lead 100 substantially similar to lead 90 of FIG. 9. Lead 100, however includes ring electrodes 104, 106 that provide electrical contact surfaces to lead 100. Ring electrodes 104, 106 may be electrically coupled to specific filars within ribbon-like structure 101. In particular, as illustrated in FIG. 10, ring electrode 104 is electrically coupled to filar 102B, whereas ring electrode 106 is electrically coupled to filar 102A.

In order to achieve such electrical coupling between a given ring electrode and a filar, a conductive surface of the filar is be exposed. For example, lead body 108 is pierced in a location desired for placement of a given ring electrode. The insulative material associated with ribbon-like structure 101 is removed and insulative cladding surrounding the given filar is also removed to expose the conductive core of the filar. Color coding of the cladding material and/or insulative material associated with ribbon-like structure 101 helps to ensure that the correct filar is used. The given ring electrode is then welded, crimped, or the like, to provide electrical coupling between the ring electrode and the exposed conductive core of the filar. In this manner, ring electrodes 104, 106 can be electrically coupled to specific filars within ribbon-like structure 101.

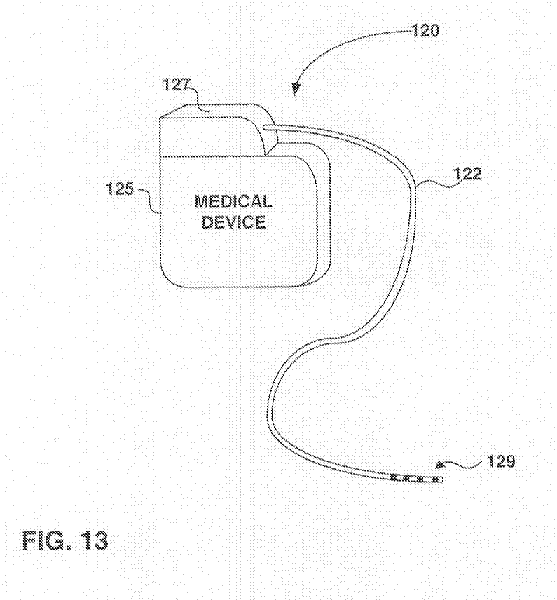

In some cases, the same electrode is electrically coupled to different filars so as to provide redundant electrical paths between the given electrode and a proximal end (not shown in FIG. 10) of lead 100. One example of a proximal end of a medical lead as described herein is illustrated in FIG. 13. The proximal end generally refers to the end of the medical lead configured for attachment to a medical device. In other cases, each ring electrode is electrically coupled to two or more filars so as to provide redundant electrical paths between the electrode and the proximal end of the lead. In any case, by forming the lead using a ribbon-like structure 101 according to the present invention, improvements to manufacturability are achieved, and improved characteristics of lead 100 are also achieved as described herein.

FIG. 11 is a schematic diagram of a coil conductor of a medical device positioned on an electrode assembly according to an embodiment of the present invention. As illustrated in FIG. 11, a coil conductor 200 according to an exemplary embodiment of the present invention includes two conductive filars 202 positioned within an insulative material 204, for example, as described above. Coil conductor 200 is wound to be formed so that an inner portion 206 of the insulative material 204 forms a conductor lumen 208. An electrode assembly 210 of a medical device lead includes two electrodes 212, for example, that are positioned on an electrode head 215 and separated by an insulative material 214, with each electrode 212 having a corresponding electrode tab 216 (only one is shown in FIG. 11) extending outward from a proximal end of electrode 212 and along an electrode shaft 218 for electrically connecting one of electrodes 212 to one of the conductive filars 202 of coil conductor 200. The electrode shaft 218 is positioned proximal from and has a diameter that is less than electrode head 215.

Although the exemplary coil conductor 200 of FIG. 11 is shown having two conductive filars 202 utilized in combination with electrode assembly 210 shown having two electrodes 212, it is understood that coil conductor 200 and electrode assembly 210 may include any desired number of filars and corresponding electrodes 212, respectively.

FIG. 12 is a partial schematic diagram of a coil conductor positioned on an electrode assembly according to an embodiment of the present invention. As illustrated in FIGS. 11 and 12, coil conductor 200 is formed with lumen 208 having a lumen diameter to enable electrode shaft 218 can be positioned within lumen 208 of coil conductor 200 with inner portion 206 coming in contact with electrode shaft 218. A portion of insulative material 204 of coil conductor 200 is removed, using laser etching, for example, to form a cut-out portion 220 in insulative material 204 so that one of the filars 202 is exposed at cut-out portion 220 of insulative material 204. The exposed portion of the filar 202 can therefore be electrically connected to a corresponding one of electrodes 212 by being welded to an associated electrode tab 216 at cut-out portion 220 (only one such connection is shown in FIGS. 11 and 12). Coil conductor 200 continues to extend around electrode shaft 218 so that the other one of filars 202 can similarly be electrically connected to the other one of electrodes 212 by having insulative material 204 removed to similarly expose the other one of filars 202 at another cut-out portion 220 so that the other one of filars 202 can be electrically connected with the other associated electrode tab 216 (not shown).

Since the diameter of electrode shaft 218 is known, coil conductor 200 can be formed so that the cut-out portion 220 for exposing one of filars 202 is spaced a predetermined distance from the other cut-out portion 220 for exposing the other one of the filars 202 to enable the two cut-out portions 220 to be aligned with the appropriate one of electrode tabs 216 once electrode shaft 218 is positioned within lumen 206 of coil conductor 200. Once electrode shaft 218 is positioned within lumen 206 of coil conductor 200 so that the cut-out portions 220 are aligned with the electrodes tabs 216, the filars 202 are electrically coupled to the electrode tabs 216 using known welding or other techniques.

FIG. 13 is a conceptual perspective view of a medical device system including a medical device coupled to a lead according to an embodiment of the present invention. As illustrated in FIG. 13, according to an exemplary embodiment of the present invention, a medical device system 120 includes a medical device housing 125 having a connector module 127 that electrically couples a proximal end of a lead 122 to various internal electrical components of medical device housing 125. In accordance with the invention, medical lead 122 incorporates one or more of the features described herein, including a set of two or more conductive filars disposed substantially parallel to one another within an insulative material, wherein the insulative material and the set of conductive filars are coiled to a define a lumen, as described above.

Medical device system 120 may include any of a wide variety of medical devices that include one or more medical lead(s) 122 and circuitry coupled to the medical lead(s) 122. By way of example, medical device system 120 may take the form of an implantable cardiac pacemaker that provides therapeutic stimulation to the heart. Alternatively, medical device system 120 may take the form of an implantable cardioverter, an implantable defibrillator, or an implantable cardiac pacemaker-cardioverter-defibrillator (PCD). Medical device system 120 may deliver pacing, cardioversion or defibrillation pulses to a patient via electrodes 129 disposed on distal ends of one or more lead(s) 122. In other words, lead 122 may position one or more electrodes with respect to various cardiac locations so that medical device system 120 can deliver pulses to the appropriate locations.

The various embodiments of the invention may provide one or more advantages. For example, by grouping multiple filars together in the insulative material, as described above, manufacture of medical leads can be simplified and improved, particularly for large scale production of multi-filar medical leads. In addition, the invention may achieve improved lead manufacturing characteristics. For example, a multi-conductor ribbon coiled medical lead according to the present invention provides for improved electrical coupling to an implanted medical device by fixedly positioning the ends of the multiple conductors relative to each other so that once the lead is properly positioned, each end of the conductor is positioned at a desired location to enable the lead connections to be made simultaneously, rather than each conductor being individually positioned and connected to electrodes. As a result, the electrical connection of the ends of the multiple conductors to the desired electrodes is simplified.

In addition, embedding the filars in insulative material allows for lead characteristics to be defined by the insulative material, such as improved lead stiffness, strength or rigidity. One or more rigid materials such as fiber cores may be embedded in the insulative material to improve such mechanical characteristics of the lead. Also, different conductive materials may be used for the different filars, e.g., so that the different conductive filars can be used for different purposes such as sensing, stimulation, high-voltage stimulation, and so forth. Color coding of cladding around the filars may also be used to help distinguish the filars from one another. Cladding around the filers may also provide redundant insulation to the conductive cores of the filers.

In certain embodiments, a wound polymer coated multi-conductor coil may be selectively bonded to the inner diameter of the lead body to enhance the torque and pushability of the assembled lead. If the insulation surrounding the filars is of the same material as the lead body, a heat bonding method may be utilized to assemble and secure the wound coil to the lead body.

It is understood that the present invention is not limited for use in pacemakers, cardioverters of defibrillators. Other uses of the leads described herein may include uses in patient monitoring devices, or devices that integrate monitoring and stimulation features. In those cases, the leads may include sensors disposed on distal ends of the respective lead for sensing patient conditions.

Also, the leads described herein may be used with a neurological device such as a deep-brain stimulation device or a spinal cord stimulation device. In those cases, the leads may be stereotactically probed into the brain to position electrodes for deep brain stimulation, or into the spine for spinal stimulation. In other applications, the leads described herein may provide muscular stimulation therapy, gastric system stimulation, nerve stimulation, lower colon stimulation, drug or beneficial agent dispensing, recording or monitoring, gene therapy, or the like. In short, the leads described herein may find useful applications in a wide variety medical devices that implement leads and circuitry coupled to the leads.

Various embodiments of the invention have been described. These and other embodiments are within the scope of the following claims.

1. A medical device lead comprising:
a first insulative material; and
a plurality of substantially parallel conductive filars extending from a proximal end to a distal end within the first insulative material.

2. The medical device lead of claim 1, wherein the first insulative material and the plurality of conductive filars are coiled to a define a lumen.

3. The medical device lead of claim 1, further comprising a plurality of second insulative materials, each insulative material of the plurality of second insulative materials positioned about a single conductive filar of the plurality of conductive filars.

4. The medical device lead of claim 3, wherein each of the plurality of second insulative materials is formed having a predetermined identifier associated with the corresponding single conductive filar.

5. The medical device lead of claim 1, further comprising a rigid material disposed substantially parallel to the plurality of conductive filars within the insulative material.

6. The medical device lead of claim 2, further comprising a first opening formed by the first insulative material spaced distally from a second opening formed by the first insulative material, the first opening exposing a first conductive filar of the plurality of conductive filars within the lumen and the second opening exposing a second conductive filar of the plurality of conductive filars at a predetermined location relative to the first opening within the lumen.

7. A medical device lead comprising:
a first insulative material;
a plurality of substantially parallel conductive filars extending from a proximal end to a distal end within the first insulative material, the first insulative material and the plurality of conductive filars coiled to a define a lumen;
an electrode assembly having a plurality of electrodes, each electrode of the plurality of electrodes having a corresponding tab extending proximally from the proximal end; and
a first opening formed by the first insulative material spaced distally from a second opening formed by the first insulative material, the first opening exposing a first conductive filar of the plurality of conductive filars within the lumen and the second opening exposing a second conductive filar of the plurality of conductive filars at a predetermined location relative to the first opening within the lumen, the first opening being positioned adjacent to the tab associated with a first electrode of the plurality of electrodes and the second opening is simultaneously positioned adjacent to the tab associated with a second electrode of the plurality of electrodes when the electrode assembly is positioned within the lumen.

8. The medical device lead of claim 7, further comprising a plurality of second insulative materials, each insulative material of the plurality of second insulative materials positioned about a single conductive filar of the plurality of conductive filars.

9. The medical device lead of claim 8, wherein each of the plurality of second insulative materials is formed having a predetermined identifier associated with the corresponding single conductive filar.

10. The medical device lead of claim 7, further comprising a rigid material disposed substantially parallel to the plurality of conductive filars within the insulative material.

11. A medical device system comprising:
a medical device housing having a connector block;
an elongated medical device lead electrically coupled to the medical device housing via the connector block and having a first insulative material; and
a plurality of substantially parallel conductive filars extending from a proximal end to a distal end within the first insulative material.

12. The medical device system of claim 11, further comprising a plurality of second insulative materials, each insulative material of the plurality of second insulative materials positioned about a single conductive filar of the plurality of conductive filars.

13. The medical device system of claim 12, wherein each of the plurality of second insulative materials is formed having a predetermined identifier associated with the corresponding single conductive filar.

14. The medical device system of claim 11, further comprising a rigid material disposed substantially parallel to the plurality of conductive filars within the insulative material.

15. The medical device system of claim 11, further comprising a first opening formed by the first insulative material spaced distally from a second opening formed by the first insulative material, the first opening exposing a first conductive filar of the plurality of conductive filars and the second opening exposing a second conductive filar of the plurality of conductive filars at a predetermined location relative to the first opening.

16. The medical device system of claim 11, wherein the first insulative material and the plurality of conductive filars are coiled to a define a lumen, the medical device system further comprising:
an electrode assembly having a plurality of electrodes, each electrode of the plurality of electrodes having a corresponding tab extending proximally from the proximal end; and
a first opening formed by the first insulative material spaced distally from a second opening formed by the first insulative material, the first opening exposing a first conductive filar of the plurality of conductive filars within the lumen and the second opening exposing a second conductive filar of the plurality of conductive filars at a predetermined location relative to the first opening within the lumen, the first opening being positioned adjacent to the tab associated with a first electrode of the plurality of electrodes and the second opening is simultaneously positioned adjacent to the tab associated with a second electrode of the plurality of electrodes when the electrode assembly is positioned within the lumen.

17. The medical device system of claim 16, further comprising a plurality of second insulative materials, each insulative material of the plurality of second insulative materials positioned about a single conductive filar of the plurality of conductive filars.

18. The medical device system of claim 17, wherein each of the plurality of second insulative materials is formed having a predetermined identifier associated with the corresponding single conductive filar.

19. The medical device system of claim 18, further comprising a rigid material disposed substantially parallel to the plurality of conductive filars within the insulative material.
